(19) United States
(12) Patent Application Publication
(10) Pub. No.: US 2026/0263173 A1
(43) Pub. Date: Sep. 10, 2026

(54) SURGICAL ROBOTIC SYSTEM AND METHOD FOR DETECTING DETACHMENT OF INSTRUMENTS

(71) Applicant: Covidien LP, Mansfield, MA (US)

(72) Inventors: Brock Kopp, Colorado Springs, CO (US); Haralambos P. Apostolopoulos, Westminster, CO (US); Christopher T. Tschudy, Arvada, CO (US); Andrew W. Zeccola, Salem, MA (US); Hannah S. Nichols, Westminster, CO (US); Alejandro J. Lepervanche, Medford, MA (US); Connor D. Roberts, Arvada, CO (US); Jason D. Kerwood, Broomfield, CO (US); Dylan R. Kingsley, Broomfield, CO (US); Jessica B. Arehart, Boulder, CO (US); James J. Butch, Denver, CO (US)

(21) Appl. No.: 19/165,732

(22) PCT Filed: Mar. 7, 2024
(86) PCT No.: PCT/IB2024/052220

§ 371 Date: Sep. 16, 2025

Related U.S. Application Data
(60) Provisional application No. 63/452,719, filed on Mar. 17, 2023.

(51) Int. Cl. A61B 34/30; A61B 17/00; A61B 18/00; A61B 18/14; A61B 90/00

(52) U.S. Cl. CPC A61B 34/30; A61B 18/1442; A61B 2017/00477; A61B 2018/00898; A61B 2018/1455; A61B 2090/066

(57) ABSTRACT

A surgical robotic system includes a surgical instrument having an end effector, at least one coupler, when rotated configured to actuate at least one function of the end effector, and a first connector. The system also includes an electrosurgical generator configured to output electrosurgical energy for energizing the end effector. The system further includes an instrument drive unit having at least one motor, a torque sensor configured to measure torque of the at least one motor, and a second connector configured to electrically couple to the first connector. The system includes a controller configured to determine a status of a connection between the first and second connectors, activate the at least one motor to rotate the at least one coupler, and determine whether the surgical instrument is detached from the instrument drive unit based the torque of the at least one motor during activation and the status of the connection.

### CROSS REFERENCE TO RELATED APPLICATIONS

This application claims the benefit of U.S. Provisional Patent Application Ser. No. 63/452,719, filed Mar. 17, 2023, the entire content of which is incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Surgical robotic systems are used with a variety of jawed surgical instruments, such as graspers, cutters, electrosurgical vessel sealers, etc.

### SUMMARY

According to one embodiment of the present disclosure, a surgical robotic system is disclosed. The surgical robotic system includes a surgical instrument having an end effector, at least one coupler, when rotated configured to actuate at least one function of the end effector, and a first connector. The system also includes an electrosurgical generator configured to output electrosurgical energy for energizing the end effector. The system further includes an instrument drive unit having at least one motor, a torque sensor configured to measure torque of the at least one motor, and a second connector configured to electrically couple to the first connector. The system additionally includes a controller configured to determine a status of a connection between the first and second connectors, activate the at least one motor to rotate the at least one coupler, and determine whether the surgical instrument is detached from the instrument drive unit based the torque of the at least one motor during activation and the status of the connection of the first and second connectors.

Implementations of the above embodiment may include one or more of the following features. According to one aspect of the above embodiment, the end effector may include a pair of opposing jaws, at least one jaw is movable relative to another jaw. The end effector may also include a knife blade reciprocating through the pair of opposing jaws. The surgical instrument may include a first coupler configured to move the at least one jaw and a second coupler configured to reciprocate the knife. The controller may be further configured to compare the torque of the at least one motor during activation to a threshold. The controller may be also configured to determine the surgical instrument is detached from the instrument drive unit in response to the torque being below the threshold. The controller may be additionally configured to output an alert in response to determining the surgical instrument is detached from the instrument drive unit.

According to another embodiment of the present disclosure, a method for detecting detachment of a surgical instrument from a robotic arm is disclosed. The method includes determining a status of a connection between a first connector of a surgical instrument and a second connector of an instrument drive unit coupled to the instrument. The method also includes activating, by a controller, at least one motor of the instrument drive unit to rotate at least one coupler of the instrument. The method further includes measuring torque of the at least one motor during activation at a torque sensor. The method additionally includes determining, at the controller, whether the surgical instrument is detached from the instrument drive unit based the torque of the at least one motor during activation and the status of the connection of the first and second connectors.

Implementations of the above embodiment may include one or more of the following features. According to one aspect of the above embodiment, the method may also include comparing the torque of the at least one motor during activation to a threshold. The method may further include determining, at the controller, the surgical instrument is detached from the instrument drive unit in response to the torque being below the threshold. The method may additionally include outputting an alert in response to determining the surgical instrument is detached from the instrument drive unit.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a cross-sectional view of a distal end portion the surgical instrument according to an embodiment of the present disclosure;

FIG. 8 is a cross-sectional view of a proximal end portion the surgical instrument according to an embodiment of the present disclosure; and

FIG. 9 is a method for detecting detachment of the surgical instrument from the instrument drive unit of FIG. 6 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

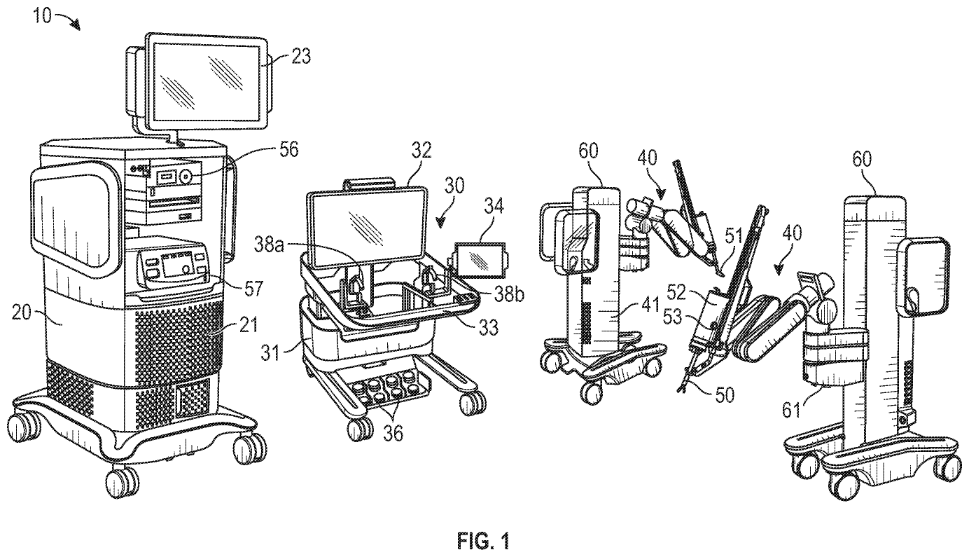

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBeeR (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

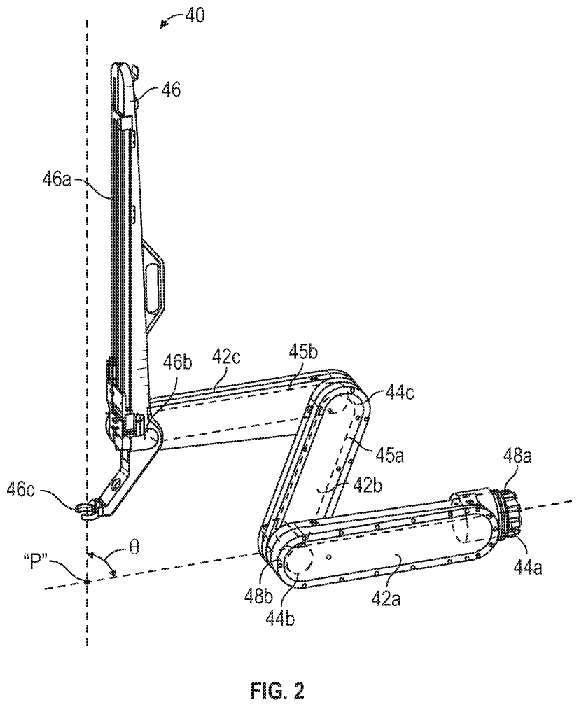

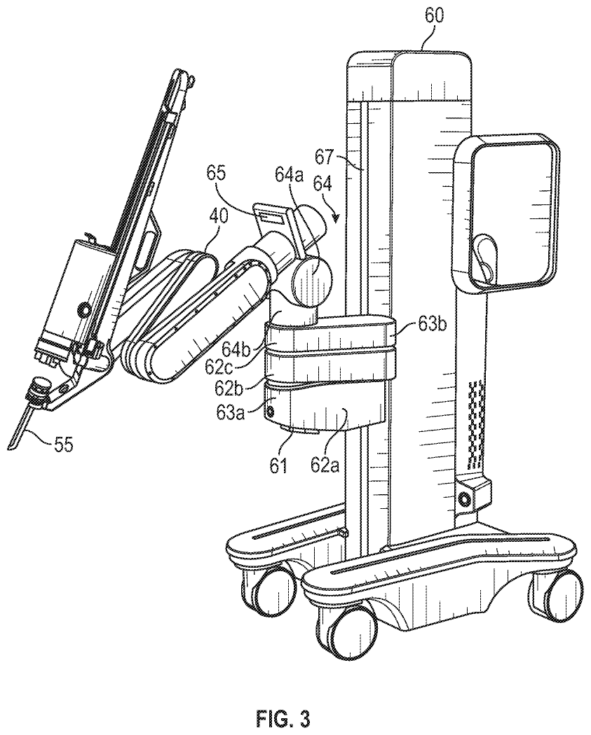

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 65 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 140 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

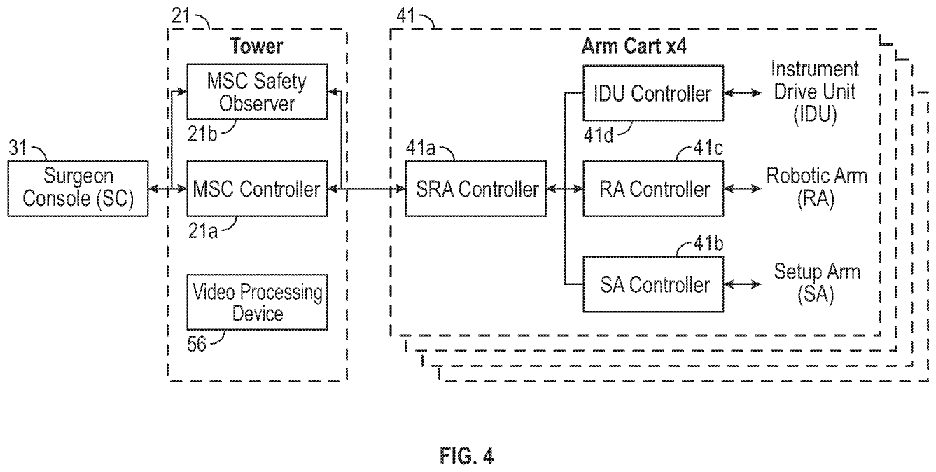

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

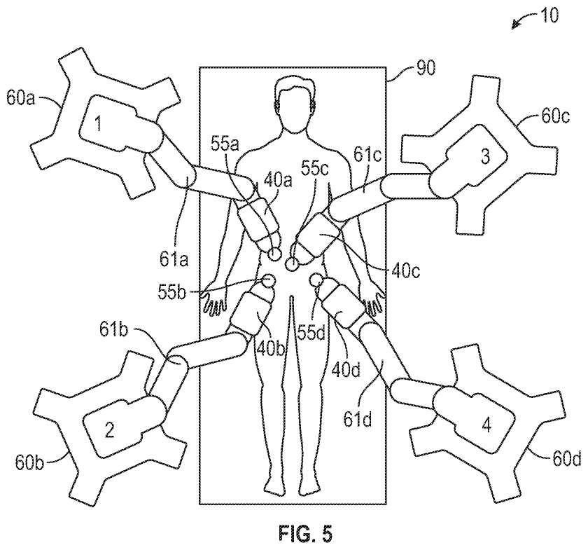

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

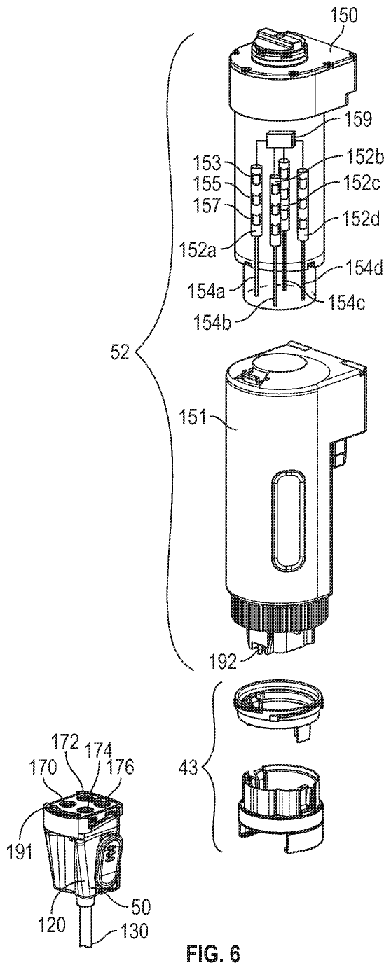

With reference to FIG. 6, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

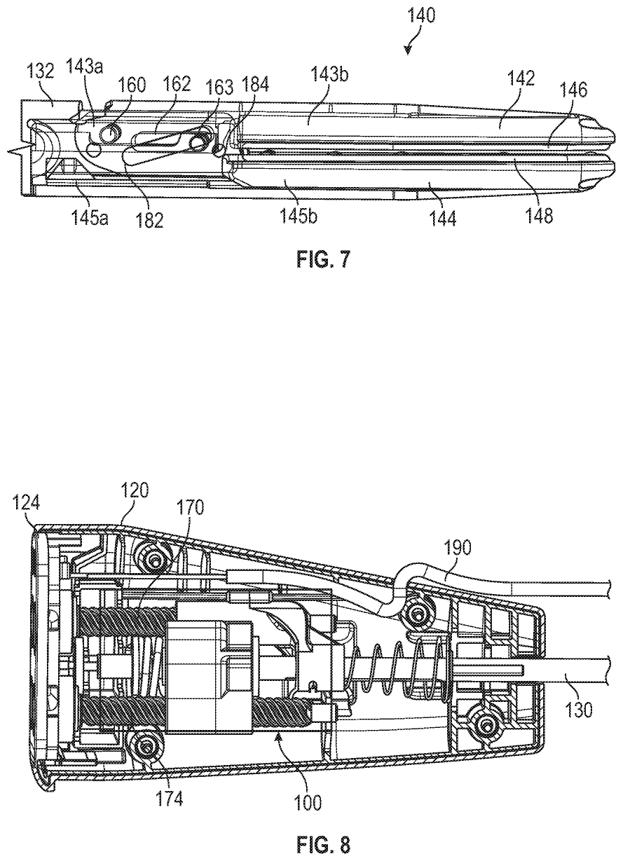

The IDU 52 includes a motor pack 150 and a sterile barrier housing 151. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 140 (FIG. 7).

Each of the motors 152a, 152b, 152c, 152d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The sensor 157 may be any device that provides a sensor signal indicative of the number of rotations of the motor 152a, such as a mechanical encoder or an optical encoder. Parameters which are measured and/or determined by the sensor 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

Referring to FIGS. 6-8, the instrument 50 includes the housing 120, a shaft 130 extending distally from housing 120, and end effector 140 extending distally from shaft 130.

A gearbox assembly 100 disposed within housing 120 and operably associated with end effector 140. Housing 120 of instrument 50 is configured to selectively couple to IDU 52 of robotic, to enable motors 152a, 152b, 152c, 152d of IDU 52 to operate the end effector 140 of the instrument 50. Housing 120 of instrument 50 supports a drive assembly that is mechanically actuated by the motors 152a, 152b, 152c, 152d of the IDU 52. Drive assembly of instrument 50 may include any suitable electrical and/or mechanical component to effectuate driving force/movement.

Instrument 50 is described herein as an articulating electrosurgical forceps configured for use with the robotic surgical system 10. However, the aspects and features of instrument 50 provided in accordance with the present disclosure, detailed below, are equally applicable for use with other suitable surgical instruments and/or in other suitable surgical systems.

With reference to FIG. 7, the instrument 50 includes an end effector 140 having first and second jaw members 142, 144, respectively. Each jaw member 142, 144 includes a proximal flange portion 143a, 145a and a distal body portion 143b, 145b, respectively. Distal body portions 143b, 145b define opposed tissue-contacting surfaces 146, 148, respectively. Proximal flange portions 143a, 145a are pivotably coupled to one another about a pivot 160 and are operably coupled to one another via a cam-slot assembly 162 including a cam pin 163 slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced-apart position (e.g., an open position of end effector 140) and an approximated position (e.g. a closed position of end effector 140) for grasping tissue between tissue-contacting surfaces 146, 148. As an alternative to this unilateral configuration, a bilateral configuration may be provided whereby both jaw members 142, 144 are pivotable relative to one another and distal segment 132 of shaft 130.

In embodiments, longitudinally extending knife channels (not shown) are defined through tissue-contacting surfaces 146, 148, respectively, of jaw members 142, 144. In such embodiments, a knife assembly including a knife assembly 182 extending from housing 120 through shaft 130 to end effector 140 and a knife blade 184 disposed within end effector 140 between jaw members 142, 144 is provided to enable cutting of tissue grasped between tissue-contacting surfaces 146, 148 of jaw members 142, 144, respectively.

With reference to FIG. 8, the housing 120 of instrument 50 includes and a proximal face plate 124 that cooperate to enclose gearbox assembly 100 therein. Proximal face plate 124 is configured to engage the IDU 52 and a plurality (e.g., four) couplers 170, 172, 174, 176 of gearbox assembly 100 extending through the plate 124.

Gearbox assembly 100 is configured to operably interface with the IDU 52 when instrument 50 is mounted on robotic surgical system 10. That is, the motors 152a, 152b, 152c, 152d of IDU 52 selectively actuate one or more of the couplers 170-176 of gearbox assembly 100 to actuate (i.e., open and close) the jaw members 142 and 144 and reciprocate the knife assembly 182 longitudinally (i.e., proximally or distally) through the jaw members 142 and 144.

With reference to FIGS. 7 and 8, the surfaces 146, 148 are formed from an electrically conductive material (e.g., stainless steel) and coupled to an electrosurgical generator 57, which is configured to output any suitable electrosurgical energy for treating (e.g., vessel sealing) tissue grasped between the surfaces 146, 148. The generator 57 is electrically coupled to the surfaces 146, 148 through a cable 190 having one or more wires, e.g., two, each of which is coupled to one of the surfaces 146, 148.

The instrument 50 also includes a first connector 191, which may be one or more biased electrical contacts, e.g., pogo pins, or any other suitable type of electrical contacts. The IDU 52 includes a second, counterpart connector 192 (e.g., having one or more counterpart contact strips configured to engage pogo pins). The connectors 191 and 192 are configured to mate with each other to establish an electrical connection for providing transmission of data and/or power signals between the instrument 50 and the IDU 52. For a more detailed description of the components of the instrument 50 and its operation reference may be made to U.S. Pat. No. 10,722,295, filed on Jan. 20, 2016, titled “Robotic surgical assemblies and electrosurgical instruments thereof,” the entire contents of which are incorporated by reference herein.

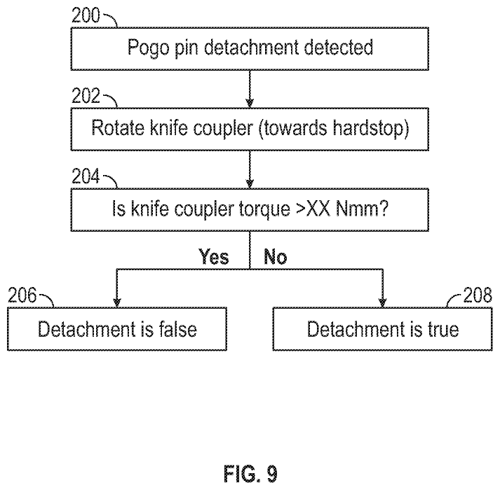

FIG. 9 shows a method for detecting detachment of the instrument 50 from the IDU 52. The method may be embodied as software instructions executable by any one or more controllers of robotic system 10 (e.g., main controller 21a, the IDU controller 41d, etc.), which is generically referred below as a controller. At step 200, the controller detects detachment of the first connector 191 from the second connector 192. This may be done by detecting a discontinuity in the electrical pathway, a voltage drop, or any other change in the signal transmission. The method also includes additional verification steps to confirm detachment of the instrument 50 by also verifying operation of the mechanical interfaces, i.e., mechanical coupling between the IDU 52 and the instrument 50.

At step 202, once the electrical discontinuity is detected, i.e., detached connectors 191 and 192, the controller verifies mechanical operation of the instrument. Verification includes commanding one of the motors 152a-d to rotate one of the couplers 170-176 and actuate a component of the end effector 140, e.g., knife assembly 182. Once commanded to rotate one of the couplers 170-176, at step 204, the controller monitors torque of the corresponding motor 152a-d at step 204 and compares the torque to a threshold indicative of actuating a component of the end effector 170-176. The threshold may be any minimum value measured by the torque sensor 155 indicative of one of the motors 152a-d moving a corresponding coupler 170-176.

If the measured torque is above the threshold, then at step 206 the controller determines that the instrument 50 is still mechanically engaged with the IDU 52 as one of the couplers 170-176 was moved by the corresponding motor 152a-d. If the measured torque is below the threshold, e.g., 0, then at step 208 the controller confirms that the instrument 50 is detached from the IDU 52 since both, the electrical interface (i.e., connectors 191 and 192) and mechanical interface (i.e., couplers 170-176) are severed. Following step 208, the controller may output an alert on one of the monitors and/or provide the alert via audio, haptic or any other suitable feedback that the instrument 50 is disconnected.

Using hard stops to verify connection may be used in any powered surgical instrument, since a similar implementation could be used on many different instruments where a home position of a mechanism is offset from its hard stop in order to not repeatedly contact that hard stop during normal use. Exemplary instruments include power staplers using an I-Beam or similar mechanism could retract to its hardtop, powered automatic suturing instruments using needle toggle mechanisms, or instruments with other similar mechanism that deploys in a single direction and has a hard stop located close to the mechanism home position.

In further embodiments, a false coupler, i.e., a coupler that is stationary and does not actuate any component of the instrument 50. This would allow the IDU 52 coupler to engage the false coupler on the instrument 50, but because this geometry is fixed to the instrument housing it would never be possible for one of the motors 152 to rotate once engaged. This would allow the IDU 52 to attempt to rotate this motor connected to this false coupler, and if the motor does not exceed a torque threshold then the system 10 will know that there is a mechanical disconnection. If the motor exceeds the torque threshold, then the mechanical connection is still engaged.

In addition to torque monitoring, the method may also monitor position and determine whether a mechanical disconnection has occurred based on the position that the motor is able to rotate. The method would operate by rotating coupler toward hard stop, continue rotating until a torque threshold is met. The method would confirm detachment of the instrument 50 if at any point during the motor movement above the motor angular position exceeds a threshold, then infer a mechanical disconnection.

Detachment events may be logged as part of the logged data on the system 10, so that users and/or manufacturer can later go back and see when and if detachment events occurred to analyze the event.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a surgical instrument including:
an end effector;
at least one coupler, when rotated configured to actuate at least one function of the end effector; and
a first connector;
an electrosurgical generator configured to output electrosurgical energy for energizing the end effector;
an instrument drive unit including:
at least one motor;
a torque sensor configured to measure torque of the at least one motor; and
a second connector configured to electrically couple to the first connector;
a controller configured to:
determine a status of a connection between the first and second connectors;
activate the at least one motor to rotate the at least one coupler; and
determine whether the surgical instrument is detached from the instrument drive unit based the torque of the at least one motor during activation and the status of the connection of the first and second connectors.

2. The surgical robotic system according to claim 1, wherein the end effector includes a pair of opposing jaws, at least one jaw is movable relative to another jaw.

3. The surgical robotic system according to claim 2, wherein the end effector includes a knife blade reciprocating through the pair of opposing jaws.

4. The surgical robotic system according to claim 3, wherein the surgical instrument includes a first coupler configured to move the at least one jaw and a second coupler configured to reciprocate the knife.

5. The surgical robotic system according to claim 1, wherein the controller is further configured to compare the torque of the at least one motor during activation to a threshold.

6. The surgical robotic system according to claim 5, wherein the controller is further configured to determine the surgical instrument is detached from the instrument drive unit in response to the torque being below the threshold.

7. The surgical robotic system according to claim 6, wherein the controller is further configured to output an alert in response to determining the surgical instrument is detached from the instrument drive unit.

8. A method for detecting detachment of a surgical instrument from a robotic arm, the method comprising:
determining a status of a connection between a first connector of a surgical instrument and a second connector of an instrument drive unit coupled to the instrument;
activating, by a controller, at least one motor of the instrument drive unit to rotate at least one coupler of the instrument;
measuring torque of the at least one motor during activation at a torque sensor; and
determining, at the controller, whether the surgical instrument is detached from the instrument drive unit based the torque of the at least one motor during activation and the status of the connection of the first and second connectors.

9. The method according to claim 8, further comprising:
comparing the torque of the at least one motor during activation to a threshold.

10. The method according to claim 9, further comprising:
determining, at the controller, the surgical instrument is detached from the instrument drive unit in response to the torque being below the threshold.

11. The method according to claim 10, further comprising:
outputting an alert in response to determining the surgical instrument is detached from the instrument drive unit.
